(19) United States
(12) Patent Application Publication
(10) Pub. No.: US 2010/0227797 A1
(43) Pub. Date: Sep. 9, 2010

(54) USE OF CYCLOLIGNANS FOR THE TREATMENT OF TYPE 2 DIABETES AND AS CONTRACEPTIVES

(75) Inventors: Magnus Axelson, Hässelby (SE); Olof Danielsson, Bromma (SE); Mario-Alexander Economou, Bromma (SE); Olle Larsson, Taby (SE)

Correspondence Address: MORRISON & FOERSTER LLP, 425 MARKET STREET, SAN FRANCISCO, CA, 94105-2482, US

(73) Assignee: AXELAR AB, Stockholm (SE)

(21) Appl. No.: 12/279,986

(22) PCT Filed: Feb. 23, 2007
(86) PCT No.: PCT/SE2007/050115

§ 371 Date: May 21, 2010

Related U.S. Application Data
(60) Provisional application No. 60/776,195, filed on Feb. 24, 2006.

(30) Foreign Application Priority Data
Feb. 24, 2006 (SE) 0600424-6

(51) Int. Cl. A61K 38/28; A61P 3/10; A61P 27/06; A61P 37/06; A61P 15/18; A61P 13/12; A61K 31/365; A61K 31/567

(52) U.S. Cl. 514/4; 514/473; 514/171

(57) ABSTRACT

There is disclosed use of certain cyclolignans for prophylaxis or treatment of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection and corneal chemical burns; and for contraception. Preferred compounds are picropodophyllin, deoxypicropodophyllin and anhydropicropodophyllol. There is also described a method of treatment of an eye disease.

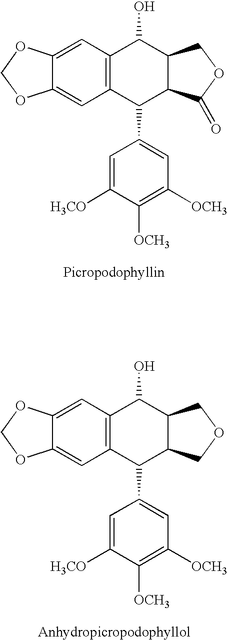

The present invention relates to the use of certain cyclolignans for prophylaxis or treatment of type 2 diabetes mellitus and its associated conditions retinopathy and nephropathy; for prophylaxis or treatment of macular degeneration and similar ocular diseases such as retinopathy of prematurity, central retinal vein occlusion, branch retinal vein occlusion, rubeotic glaucoma, thyroid eye disease, corneal graft rejection and corneal chemical burns. Moreover the present invention relates to use of certain cyclolignans for contraception. The present invention further relates to a method of treatment of an eye disease.

### BACKGROUND OF THE INVENTION

The incidence of diabetes mellitus type 2, which is caused by increased insulin resistance is increasing all over the world but its pathogenesis is not known. Although elevated levels of blood glucose may be pharmacologically relatively well treated, some of its associated conditions or complications such as nephropathy and retinopathy (an ocular disease causing loss of vision) lack efficient prophylaxis and treatment.

An eye disease, which in many aspects is similar to the latter, is macular degeneration. This disease is usually age-related. Macular degeneration is actually the leading cause of blindness because it frequently damages the retina. Today there are no treatment strategies to prevent or reduce the progress of this disease efficiently.

Other similar eye diseases are retinopathy of prematurity (ROP), a potentially blinding eye disorder that affects premature infants weighing less than 1.3 kg and are born before week 31 of gestation; central or branch retinal vein occlusion (CRVO and BRVO), that are caused by a blood clot in the central or in a branch of the retinal veins reducing or stopping the blood flow from the retina; rubeotic glaucoma, which is secondary to diabetes or CRVO; and thyroid eye disease (TED), a complication of Graves' disease and characterized by inflammation of orbital soft tissues, cornea and optic nerves. In addition, corneal graft rejection is a common complication of corneal transplantation and corneal chemical burn is a condition where the cornea is traumatised by acid or alkali. There are no effective treatments to prevent or reduce the progress of these diseases and conditions today.

Unplanned and unwanted pregnancies are linked to severe risks both from a medical and a social point of view and alternative safe contraceptive techniques with a minimum of side effects are continuously being searched for.

Growth factors belong to a group of biologically potent substances, which generally promote cell growth. These compounds can interact in complicated ways with each other and are suspected to be involved in the development of many diseases and physiological processes. One such growth factor is the insulin-like growth factor-1 (IGF-1), which has been shown to play a significant role in cancer development and in certain endocrine disorders such as acromegaly.

A number of cyclolignans have been shown to inhibit the cellular signalling of the latter growth factor by interfering with its receptor.

### PRIOR ART

Type 2 diabetes mellitus is a common disease. The number of adults with diabetes in the world was about 170 millions in the year 2000 and is rising. The pathogenesis of type 2 diabetes mellitus is complex, involving progressive development of insulin resistance and relative deficiency in insulin secretion, leading to overt hyperglycaemia. Currently available therapies for type 2 diabetes include insulin and various oral agents, such as sulfonyl ureas, metformin and α-glucosidase inhibitors. These agents are used as monotherapy in newly diagnosed patients. In patients where the disease is more advanced, such drugs are frequently used in combination to achieve better glycemic control (DeFronzo R A, Ann Intern Med 1999; 131(4):281-303). Most of the above oral agents suffer from inadequate long-term efficacy as monotherapy and also a number of adverse effects. Thus, there is a high demand for new oral antidiabetic drugs (Schernthaner G H, et al., Scand J Clin Lab Invest Suppl 2005; 240:30-40). Advanced diabetes mellitus often leads to feared complications such as nephropathy and diabetic retinopathy (Cordain L, et al., Comp Biochem Physiol A Mol Integr Physiol. 2003; September, 136(1):95-112; Frystyk J, Horm Metab Res., 2005 April, 37 Suppl 1:44-8). The latter complication is particularly feared since it frequently leads to blindness.

An associated eye disease is macular degeneration, particularly exudative age-related macular degeneration (and related choroidal diseases), which can also cause lesions in the retina. The exudative form of age-related macular degeneration is characterized by growth of abnormal vessels that invade the subretinal space, often leading to exudation and haemorrhage. This will lead to damage of photoreceptors and loss of central vision, and after several months, the vessels are largely replaced by fibrovascular scar tissue. Although the pathogenesis of macular degeneration is unknown, a number of growth factors are believed to be, at least partly, involved in the progression of the disease. Age-related macular degeneration is the major cause of blindness in people older than 55 years in the developed world and no efficient treatment is available. It is expected to develop in almost 1 million people in the United States within next five years making it a major public health issue. (Kim R W, et al., Opthalmol Clin. 2004; Fall; 44(4):41-50). Retinopathy of prematurity (ROP) occurs when abnormal blood vessels throughout the retina cause haemorrhage and scarring resulting in a retinal detachment and visual impairment and blindness. This severe eye disease is affecting about 15,000 newborn infants every year in the United States. Similar eye diseases are the central and branch retinal vein occlusion (CRVO and BRVO) affecting the retinas of people more than 45 years of age. Main risk factors are hypertension, hypercholesterolemia and diabetes. The most severe complications are pathological retinal vessels, macular edema and secondary glaucoma which can lead to blindness, being the second most frequent cause of this in elderly in the industrialised world (Hattenbach LO, et al., Opthalmologica 1999; 213(6):360-6). Rubeotic glaucoma is frequently associated with proliferative diabetic retinopathy and central retinal vein occlusion. The pathogenesis is not known, but vessels in the angle of the eye cause obstruction to fluid egress via the trabecular meshwork being the primary outflow pathway of the eye (Sivak-Callcott J A et al., Opthalmology 2001; October; 108(10):1767-76). Thyroid eye disease (TED) is an autoimmune disease characterized by enlargement of the extraocular muscles and expansion of retrobulbar fatty/connective tissue compartment. These changes cause exopthalmos, periorbital swelling and venous congestion. An involvement of cornea and optic nerves may also occur. The pathogenesis of TED is almost certainly multifactorial, also involving cytokines and growth factors (Heufelder A E, J Endocrinol Invest 1997; 20(Suppl 7):50-52). Over 40,000 corneal transplants were performed in the 1990s in the United States and Canada, but the 5-year postoperative failure rate is about ⅓, mainly due to corneal graft rejection. This is caused by a specific immunologic response of the host to the donor corneal tissue. Preexisting and outgrowth of corneal stromal blood vessels are strong risk factors for this immune rejection (Cursiefen C, et al., Cornea 2003; 22:273-281). Chemical corneal burns can be provoked from acid and alkali, representing 10% of all eye injuries. The condition can lead to blindness mainly caused by development of the abnormal blood vessels in the cornea (Chang J H, et al. Curr Opin Opthalmol 2001; 12:242-249).

The pathogenesis (and/or progression) of the described eye diseases and conditions are largely unknown, but a number of growth factors are believed to be, at least partly, involved in the progression of them.

Approximately 1 million women in the world are becoming pregnant every day. Half of these pregnancies are believed to be unplanned and half of the latter unwanted. Every day about 150 000 abortions are being performed. Unwanted pregnancies are linked to harmful effects or risks both from a medical and a social point of view. The risk of morbidity and death associated with therapeutic abortion is greater than that associated with hormonal contraceptive use. An ideal method of contraception has not yet been developed. Alternative safe contraceptive techniques with a minimum of side effects are therefore continuously being searched for.

A number of growth factors are believed to be involved in the reproductive process, for example promoting the follicular development, embryo development, implantation and in maintaining pregnancy (Pinto A B, et al., Hum Reprod 2002; February; 17(2):457-62).

Growth factors belong to a group of biologically potent substances, which generally promote cell growth. These compounds can interact in complicated ways with each other and are suspected to be involved in the development of many diseases and physiological processes. One such growth factor is IGF-1, whose receptor is a member of the tyrosine kinase receptor family. This family also includes receptors of insulin, epidermal growth factor (EGF), nerve growth factor (NGF) and platelet-derived growth factor (PDGF). The signal mediated by these receptors after stimulation can be affected by substances interfering with for example the activity of their tyrosine kinases, the latter therefore being attractive pharmacological targets (Levizki A, et al., Science, 1995; 267: 1782-1788).

In PCT/WO02/102804/A1 and PCT/WO2004/055022 (A Girnita, L Girnita, F d Prete, A Bartolazzi, O Larsson and M Axelson: Cyclolignans as inhibitors of the insulin-like growth factor-1 receptor and malignant cell growth. Cancer Research, 64, 236-242, 2004), there is disclosed the use of specific cyclolignans for inhibition of the IGF-1 receptor without affecting the insulin receptor. The compounds could be used for treatment of IGF-1R dependent diseases, particularly cancer. One of these cyclolignans was picropodophyllin, which had previously been considered to possess little or no biological activity as stated by Y Damayanthi and J W Lown in Current Medicinal Chemistry (1998; 5:205-252).

### OBJECTS OF THE INVENTION

The object of the invention is to provide new methods for prophylaxis or treatment of diabetes mellitus type II and its associated conditions such as retinopathy and nephropathy and for prophylaxis or treatment of macular degeneration and similar ocular diseases including retinopathy of prematurity, central retinal vein occlusion, branch retinal vein occlusion, rubeotic glaucoma, thyroid eye disease, corneal graft rejection and corneal chemical burns. Another object of the present invention is to provide a method for contraception.

### BRIEF DESCRIPTION OF THE DRAWINGS

FIG. 1 shows the structural formulas of the compounds picropodophyllin and anhydropicropodophyllol.

FIG. 2 shows the structural formulas of the compounds deoxypicropodophyllin and deoxyanhydropicropodophyllol.

### DESCRIPTION OF THE INVENTION

When treating mice with certain cyclolignans, it was discovered that the blood levels of glucose decreased. Such a result was unexpected, since the action of IGF-1 has been said to mimic that of insulin. In fact, it has even been suggested that IGF-1 per se may be used to treat diabetes mellitus. Notably, the anti-diabetic effect of the cyclolignans during a prolonged treatment period only resulted in normalization of blood glucose levels and did not result in the feared condition of hypoglycemia. The results showed that the cyclolignans can be used to lower blood glucose levels and thereby prevent or treat type 2 diabetes mellitus, which is caused by increased insulin resistance. It is also likely that the cyclolignans therefore can have some beneficial effects on complications of this disease, such as nephropathy and retinopathy, which develop particularly in the poorly treated patients with advanced disease.

To demonstrate that this may be the case, the effects of the cyclolignans were tested on a partly similar eye disease as retinopathy, in this case macular degeneration. The latter disease is believed to develop by the involvment of a number of growth factors. The results show for the first time that treatment with a cyclolignan protects the eye from progress of macular degeneration using a mouse model. The noted protective effect of cyclolignans can also be valid for eye diseases with a pathogenesis resembling that of macular degeneration.

Another physiological condition in which a number of growth factors have been suggested to play important roles is in different reproductive processes, such as follicular and embryo development, implantation and pregnancy (Pinto A B, et al., Hum Reprod 2002; February; 17(2):457-62).

In order to test if the cyclolignans could prevent ovulation and implantation of the embryo and thereby act as a contraceptive agent, the effects of picropodophyllin were studied on mouse models. The results show that the compound prevents ovulation and has a negative effect on implantation.

In one aspect the present invention provides use of a compound according to formula I,

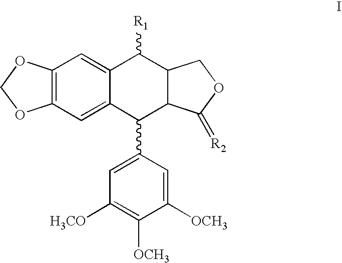

wherein R₁ is selected from the group consisting of H, OH, and an ester group, and R₂ is selected from the group consisting of O and two H, as well as pharmaceutically acceptable salts thereof, for the manufacture of a medicament for prophylaxis or treatment of at least one disease selected from the group consisting of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection and corneal chemical burns.

In another aspect the present invention provides use of a compound according to formula I, wherein R₁ is selected from the group consisting of H, OH, and an ester group, and R₂ is selected from the group consisting of O and two H, as well as pharmaceutically acceptable salts thereof, for contraception.

In one embodiment of the present invention there is provided use of a compound according to formula II,

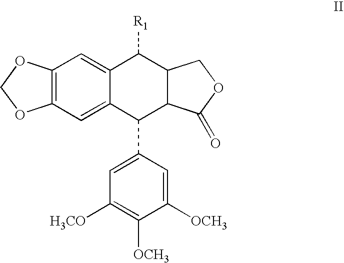

wherein R₁ is selected from the group consisting of H, OH, and an ester group, as well as pharmaceutically acceptable salts thereof, wherein the lactone ring has a cis configuration with two beta bonds, and wherein R₁ and the trimethoxyphenyl group are in alpha-position, for the manufacture of a medicament for prophylaxis or treatment of at least one disease selected from the group consisting of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection and corneal chemical burns.

Notably, compounds of the formula II have a lactone ring with a cis configuration, i.e. two beta bonds, as indicated by the solid lines. R₁ and the trimethoxyphenyl group are preferably in alpha-position, as is illustrated by dashed lines.

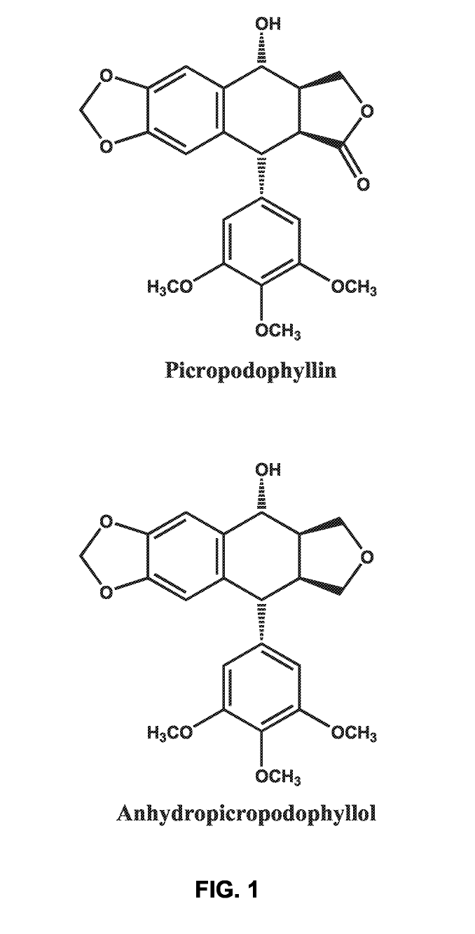

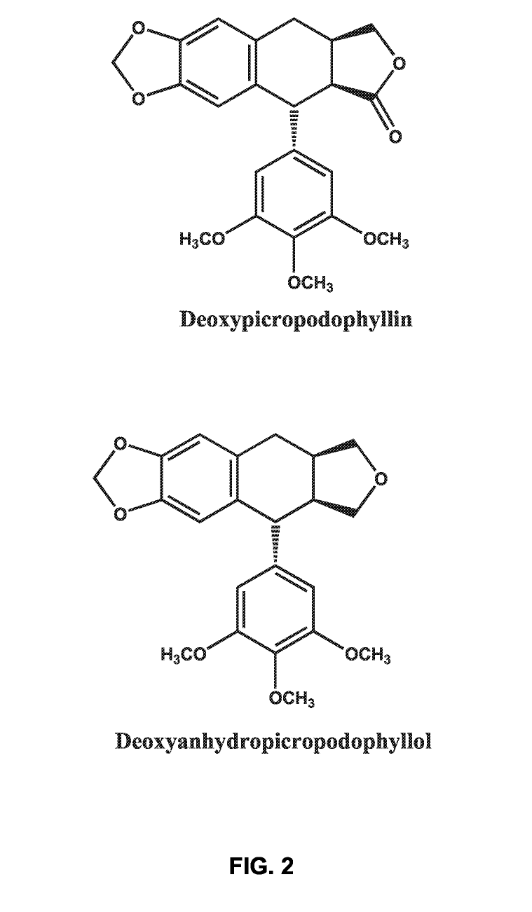

Preferred compounds are picropodophyllin (FIG. 1) and deoxypicropodophyllin (FIG. 2).

The present invention also provides use of a compound having the formula III,

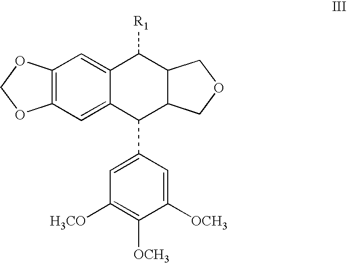

wherein R₁ is selected from the group consisting of H, OH, and an ester group, as well as pharmaceutically acceptable salts thereof, wherein the cyclo-ether ring has a cis configuration with two beta bonds, and wherein R₁ and the trimethoxyphenyl group are in alpha-position, for the manufacture of a medicament for prophylaxis or treatment of at least one disease selected from the group consisting of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection and corneal chemical burns.

Notably, compounds of the formula III have a cyclo-ether ring with a cis configuration, i.e. two beta bonds, as indicated by the solid lines. R₁ and the trimethoxyphenyl group are preferably in alpha-position, as is illustrated by dashed lines.

Preferred compounds are anhydropicropodophyllol (FIG. 1) and deoxyanhydropicropodophyllol (FIG. 2).

In particular R₁ may be an ester group. Any pharmaceutically acceptable ester group can be used. Non-limiting examples of ester groups are selected from the group consisting of OCOH, OCO(CH₂)₀₋₁₈CH₃, OCOCH(CH₃)₂, OCO(CH₂)₂COOH, OCOCH₂N(CH₃)₂, OCONHCH₂CH₃, OCOC₅NH₄ and OPO₃H₂. In one particular embodiment R₁ is OCOCH₂N(CH₃)₂. In one embodiment the ester comprises a carboxylic group or another acid group. In such a case any compound according to the present invention may be provided as a pharmaceutically acceptable salt. A person skilled in the art is able to choose a suitable pharmaceutically acceptable salt.

There is provided use of any compound mentioned above for the manufacture of a medicament for prophylaxis or treatment of diabetes mellitus type 2.

There is provided use of any compound mentioned above in combination with another drug selected from the group consisting of insulin, sulfonylureas, metformin and alpha-glucosidase inhibitors.

There is provided use of any compound mentioned above for the manufacture of a medicament for prophylaxis or treatment of nephropathy.

There is provided use of any compound mentioned above for the manufacture of a medicament for prophylaxis or treatment of diabetic retinopathy.

There is provided use of any compound mentioned above for the manufacture of a medicament for prophylaxis or treatment of macular degeneration.

There is provided use of any compound mentioned above for the manufacture of a medicament for prophylaxis or treatment of retinopathy of prematurity.

There is provided use of any compound mentioned above for the manufacture of a medicament for prophylaxis or treatment of central or branch retinal vein occlusion (CRVO or BRVO).

There is provided use of any compound mentioned above for the manufacture of a medicament for prophylaxis or treatment of rubeotic glaucoma.

There is provided use of any compound mentioned above for the manufacture of a medicament for prophylaxis or treatment of thyroid eye disease.

There is provided use of any compound mentioned above for the manufacture of a medicament for prophylaxis or treatment of corneal graft rejection.

There is provided use of any compound mentioned above for the manufacture of a medicament for prophylaxis or treatment of corneal chemical burns.

There is provided use of any compound mentioned above for contraception.

There is provided use of a compound mentioned above in combination with at least one other drug selected from the group consisting of an estrogen, a progestagen, and a prostaglandin.

There is provided use of any compound mentioned above in combination with mifepristone.

In another aspect there is provided a method of treatment of an eye disease comprising administrating a pharmaceutical composition, comprising a compound according to any of the formulas I-III in combination with a physiologically acceptable carrier.

In one embodiment there is provided a method wherein the pharmaceutical composition is administered locally in the eye.

In another embodiment there is provided a method wherein the pharmaceutical composition is administered near the eye.

Podophyllotoxin and deoxypodophyllotoxin, used as starting materials for the syntheses of the described picro derivatives, i.e. cyclolignans with a cis configuration in their lactone or ether ring, are naturally occurring in plants. For the preparation of said substances in pure form, dried and finely ground rhizomes of e.g. Podophyllum emodi or Podophyllum peltatum are extracted with organic solvents. The extract is then filtered and concentrated on silica gel. The fractions containing the substances are collected and the latter are further purified by chromatography on acid alumina and silica gel etc., and finally recrystallized.

Picropodophyllin and deoxypicropodophyllin are then prepared from purified podophyllotoxin and deoxypodophyllotoxin, respectively, using essentially identical procedures. Picropodophyllin was dissolved in 70% aqueous ethanol. To the solution was added sodium acetate and the mixture was then refluxed and stirred for 12 h. The mixture was cooled and filtered. The product (precipitate) was washed with ethyl acetate, and then purified by recrystallization from absolute ethanol essentially as described by O Buchardt et al. (J Pharmaceut Sci 1986; 75:1076-1080) or purified by chromatography on silica gel, mobile phase: hexane-ethyl acetate mixtures, and/or octadecylsilane-bonded silica, mobile phase: aqueous methanol.

The total synthesis of picropodophyllin has been described by J W Gensler et al. (J Am Chem Soc 1960; 82:1714-1727).

Anhydropicropodophyllol (picropodophyllin cyclic ether) and deoxyanhydropicropodophyllol (deoxypicropodophyllin cyclic ether) are prepared from picropodophyllin and deoxypicropodophyllin, respectively (see Gensler W J, et al., J Med Chem 1977; 20:635-644).

Briefly, picropodophyllin was first converted to tetrahydropyranylpicropodophyllin. Picropodophyllin was dissolved in the reagent dihydropyran plus some small crystals of p-toluenesulfonic acid monohydrate and the mixture was stirred for 1 h. A 3% aqueous bicarbonate solution was then added, the volatiles removed in vacuo, and the residue (containing tetrahydropyranylpicropodophyllin) was then extracted with diethyl ether and washed.

The recovered tetrahydropyranylpicropodophyllin was then reduced to tetrahydropyranylpicropodophyllol. Tetrahydropyranylpicropodophyllin was dissolved in dry diethyl ether and lithium aluminum hydride was added. After 3.5 h of stirring at room temperature, the mixture was cooled to 0° C. and then carefully treated with water. The produced tetrahydropyranylpicropodophyllol was then extracted by ethyl acetate and washed with water and dried. The recovered tetrahydropyranylpicropodophyllol was then converted to tetrahydropyranylanhydropicropodophyllol by dissolving the former in pyridine and then adding p-toluenesulfonyl chloride in pyridine. The mixture was allowed to react for 4.5 h without contact with air and then some extra of p-toluenesulfonyl chloride was added and the reaction was allowed to proceed for another 4.5 h. The product tetrahydropyranylanhydropicropodophyllol was then extracted with ethyl acetate from the water phase and dried. Tetrahydropyranylanhydropicropodophyllol was hydrolyzed to anhydropicropodophyllol by dissolving the former in ethanol acidified with concentrated HCl and allowing the mixture to stand 1 h in room temperature. After neutralization with aqueous sodium bicarbonate, anhydropicropodophyllol was extracted with ethyl acetate and dried.

An alternative method for the synthesis of anhydropicropodophyllol was also tested, and this method gave higher yields of the compound than the method described above. Briefly, the tertbutyldimethylsilyl ether of picropodophyllin was first prepared by adding tert-butyldimethylsilyl (t-BDMS) chloride under N₂ to a mixture of picropodophyllin and imidazole in dimethylformamide. The yellow solution was stirred overnight at room temperature and poured into water. The derivative was purified prior to reduction of the lactone group with lithium aluminum hydride in tetrahydrofuran. The latter mixture was then stirred at room temperature for 3 hours yielding the t-BDMS derivative of picropodophyllol (having two free hydroxyl groups). To a solution of this compound in dichloromethane was added triphenylphosphine and diethyl azodicarboxylate and the mixture was then stirred at room temperature for 3 hours. The solvent was evaporated and the crude t-BDMS ether of anhydropicropodophyllol was then purified. Underivatized anhydropicropodophyllol was obtained by adding tetrabutyl ammonium fluoride to a solution of the derivative in tetrahydrofuran. The mixture was then stirred at room temperature over night. After purification, free and pure anhydropicropodophyllol was obtained as a white solid.

Deoxyanhydropicropodophyllol can be synthesized from deoxypicropodophyllin in a similar but more simple way. Briefly, the lactone group of deoxypicropodophyllin is reduced using lithium aluminum hydride in tetrahydrofuran. The mixture is stirred at room temperature for 3 hours yielding deoxypicropodophyllol (having two free hydroxyl groups). To a solution of this compound in dichloromethane are added triphenylphosphine and diethyl azodicarboxylate and the mixture is then stirred at room temperature for 3 hours. The product deoxyanhydropicropodophyllol is then obtained after purification.

As additional examples of compounds of the formulas II and III can be mentioned various esters of picropodophyllin and anhydropicropodophyllol and pharmaceutically acceptable salts thereof, which can be prepared by conventional procedures.

In case of diseases/conditions requiring additional therapy, treatment with the compounds of the invention may be combined with other treatments. For example, the compounds can be useful to sensitize cells and potentiate the effect of other treatments. The invention therefore also refers to the use of a compound of the formula I in combination with another therapy such as a pharmaceutical drug, surgery etc. As examples of drugs or therapies which can be used together with the compounds of the invention for the treatment of diabetes mellitus type II can be mentioned insulin and various oral agents such as sulfonylureas, metformin, α-glucosidase inhibitors and for contraception can be mentioned preparations/drugs containing estrogens and progestagens, prostaglandins and mifepristone.

The cyclolignans are valuable for prophylaxis or treatment of many diseases, such as type 2 diabetes mellitus, nephropathy and retinopathy, for prophylaxis or treatment of macular degeneration, retinopathy of prematurity, central retinal vein occlusion, branch retinal vein occlusion, rubeotic glaucoma, thyroid eye disease, corneal graft rejection and corneal chemical burns; and for contraception by interfering with different steps in the female reproductive process starting with ovulation.

An aspect of the invention refers to the use of compounds of the formula I for the preparation of a medicament for treatment of diabetes mellitus type 2 (Nam S Y, et al., Int J Obes Relat Metab Disord 1997; 21:355-359; Attia N, et al., J Clin Endocrinol Metab 1998; 83:1467-1471). Conditions which are linked to this disease as complications are nephropathy and retinopathy (Thierry van Dessel H J, et al., J Clin Endocrinol Metab 1993; 77:776-779).

Another aspect of the invention refers to the use of compounds of the formula I for the preparation of a medicament to be used for prophylaxis and treatment of macular degeneration, particularly exudative age related macular degeneration, and similar eye diseases, such as retinopathy of prematurity, central retinal vein occlusion, branch retinal vein occlusion, rubeotic glaucoma, thyroid eye disease, corneal graft rejection and corneal chemical burns. Notably, age-related macular degeneration is a major cause of blindness and no efficient treatment is available.

Yet another aspect of the invention refers to the use of compounds of the formula I for contraception by interfering with different steps in the reproductive process, for example ovulation or implantation of the embryo in the endometrium. Implantation is the most important biological process during the initiation of pregnancy. Embryo implantation is mediated by the trophoblasts, which attach to and invade the endometrium eventually leading to a mature placenta and a viable fetus. Different growth factors may play roles in the adhesive and migratory events that are considered to be crucial in the implantation process (Kabir-Salmani M, et al., J Clin Endocrinol Metab 2002; 87:5751-5759; Korgun E T, Reproduction 2003; 125:75-84).

For oral administration, the compounds can be formulated into solid or liquid preparations such as capsules, pills, tablets, troches, powders, solutions, suspensions or emulsions.

For topical application the compounds can be administered in the form of an unguent, cream, ointment, lotion, solution or a patch. For parenteral administration, the compounds may be administered as injectable dosages or by continuous intravenous infusion of a solution, suspension or emulsion of the compound in a physiologically acceptable diluent as the pharmaceutical carrier, which can be a sterile liquid, such as water, alcohols, oils, emulsions, and other acceptable organic solvents, with or without the addition of a surfactant and other pharmaceutically acceptable adjuvants.

The compounds can also be administered in the form of a depot injection or implant preparation, which may be formulated in such a manner as to permit a sustained release of the active ingredient. The invention also refers to a method of treatment of the above mentioned eye diseases (diabetes retinopathy, macular degeneration, retinopathy of prematurity, central retinal vein occlusion, branch retinal vein occlusion, rubeotic glaucoma, thyroid eye disease, corneal graft rejection and corneal chemical burns), comprising the steps of administrating a pharmaceutical composition, containing a compound having the formula I in combination with a physiologically acceptable carrier, by local treatment in or nearby the eye, for example eye drops or eye cream etc.

### EXPERIMENTAL

### Materials

### Chemicals

Picropodophyllin (99% purity) and deoxypicropodophyllin (99% purity) were synthesized from podophyllotoxin (from Sigma and other commercial sources) and deoxypodophyllotoxin (a gift from Analytecon SA, Pre Jorat, Switzerland), respectively. Anhydropicropodophyllol (99% purity) was synthesized from picropodophyllin.

### Methods

### Assay of Blood Glucose

Ten weeks old SCID mice were treated with PPP (20 mg/kg/12 h) intraperitoneally using DMSO as vehicle. Control mice were treated with vehicle only. Three mice were treated in each group. After treatment for 7 days, the mice were sacrificed 4 h after the last injections. Blood samples were taken and the concentration of glucose in the serum was determined by a dry slide technique using Vitros 950 Chemistry System.

All experiments were performed according to the ethical guidelines for laboratory animal use and approved by the institutional ethical committee.

### Laser Induction of Macular Degeneration Lesions

To induce macular degeneration lesions adult C57Bl/6 mice were anesthetized and three laser spots were placed in fundus flavimaculatus of the choroidea with a krypton red laser (614 nm, 50 mm, 0.05 second, 200 mW). The mice were then treated with PPP (20 mg/kg/12 h) intraperitoneally using DMSO as vehicle for two weeks. Control mice were treated with vehicle only. Twenty mice were treated in each group. Two weeks after the laser treatment, the animals were perfused with 3% FITC-conjugated high-molecular-weight dextran, sacrificed and choroidal flat mounts were prepared. Flat-mounts were examined with fluorescence microscopy Axioskop microscope. Macular degeneration lesions were identified as FITC-perfused vessels. Image-Pro Plus software was used to measure the area of each CNV lesion. Data were recorded as area of macular degeneration lesions (μm²).

The experiments described below on macular degeneration were performed in collaboration with Dr. A. Kvanta at S:t Eriks Eye Hospital, (Stockholm, Sweden) and those on contraception using models for ovulation and implantation of embryo were carried out in collaboration with Dr. K. Gemzell-Danielsson, Karolinska Institutet, Stockholm, Sweden.

### Experiment 1

### Effect of Picropodophyllin on Blood Glucose Levels in Mice

In this experiment, healthy SCID mice were treated with picropodophyllin by intraperitoneal injections twice daily, the dose being 20 mg/kg/12 h. The control group was treated with the vehicle only (totally per day: 20 μL DMSO). Each group included 3 mice. The results are shown in Table 1.

TABLE 1
Effect of one week picropodophyllin(PPP) treatment
on blood glucose levels in mice.

| | Glucose level in serum |
|---|---|
| Mice | (mM) |
| Controls: | |
| No. 1 | 7.5 |
| No. 2 | 6.3 |
| No. 3 | 7.2 |
| Mean: | 7.0 |
| PPP-treated: | |
| No. 1 | 4.5 |
| No. 2 | 4.5 |
| No. 3 | 5.7 |
| Mean: | 4.9 |

The results show that picropodophyllin decreased the blood glucose levels in mice suggesting a stimulated activity of insulin/insulin receptors in the picropodophyllin-treated mice compared to the controls.

### Experiment 2

### Effect of Picropodophyllin on a Macular Degeneration Animal Model

Macular degeneration is the leading cause of vision loss. Macular degeneration lesions were induced in 40 adult C57Bl/6 mice by laser. Twenty animals received intraperitoneal (i.p.) injections of picropodophyllin (PPP; 20 mg/kg/12 h) for 2 weeks. Controls received i.p. injections of vehicle. Two weeks after the laser treatment the animals were sacrificed and choroidal flat mounts were prepared. Flat-mounts were examined with fluorescence microscopy. Image-Pro Plus software was used to measure the area of each CNV lesion. The results are shown in Table 2.

TABLE 2
Effect of treatment with picropodophyllin(PPP) for 2
weeks on macular degeneration lesion area in mice.

| Treatment | Area of macular degeneration (μm²) | Significance |
|---|---|---|
| Vehicle | Mean: 2.565 | |
| PPP-treated | Mean: 1.753 | P = 0.0185* |

*Student's t-test

The results show a decrease in macular degeneration lesion area by 32% in the picropodophyllin treated group, which was statistically significant. Thus, picropodophyllin reduces macular degeneration.

### Experiment 3

### Effect of Picropodophyllin on Ovulation in a Mouse Model

Female mice were treated with picropodophyllin (PPP; 20 mg/kg/12 h, given intrapritoneally) on days −1 and 0 of mating. The vaginal plug was checked on the next morning. Six animals from each group were sacrificed 4 days after mating. The organs collected were ovaries, uterus, liver and spleen for biochemical analysis. The results are shown in Table 3.

TABLE 3
Effect of picropodophyllin (PPP) on ovulation.

| | Number of | Number of mice with | Percent ovulated |
|---|---|---|---|
| Treatment | mice | positive plug | mice ** |
| PPP | n = 36 | n = 0 | 0% |
| Vehicle | n = 6 | n = 3 | 50% |

The results show that female mice treated with picropodophyllin did not ovulate at all, while 50% of the mice treated with vehicle ovulated. The inhibition of ovulation by picropodophyllin was complete and strongly significant (P=0.005919; Fishers exact test).

### Experiment 4

### Effect of Picropodophyllin on Implantation in a Mouse Model

The animals were put for mating and the vaginal plug was checked on the next morning. Six female mice positive for vaginal plug were treated with picropodophyllin (20 mg/kg/12 h, given intraperitoneally) and six with vehicle (controls) from day 3 to 6 after the plug. They were sacrificed on day 9 after ovulation. The organs collected were ovaries, uterus, liver and spleen for biochemical analysis. The results are shown in Table 4.

TABLE 4
Effect of picropodophyllin on implantation

| Treatment | Number of mice | Number of pregnancies |
|---|---|---|
| Vehicle | n = 6 | 4 |
| PPP-treated | n = 6 | 2* |

*not statistically significant

The results strongly suggest that picropodophyllin interferes negatively with implantation of the embryo in uterus, but the number of animals were too low for obtaining statistical significance.

1. A method of treatment for a selected disease or condition, the method comprising:
administering to a subject a pharmaceutical composition comprising a compound according to formula I, or a pharmaceutically acceptable salt thereof, and a pharmaceutical adjuvant or a pharmaceutical carrier, 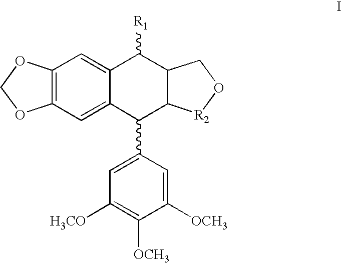
wherein the pharmaceutical composition is administered in an amount effective to prevent or reduce one or more clinical symptoms of the selected disease or condition, wherein R₁ is selected from the group consisting of H, OH, and an ester group, and R₂ is selected from the group consisting of CO or CH₂, wherein the selected disease or condition is a disease or condition selected from the group consisting of diabetes mellitus type 2, nephropathy, retinopathy, macular degeneration, retinopathy of prematurity, central retinal vein occlusion, branch retinal vein occlusion, rubeotic glaucoma, thyroid eye disease, corneal graft rejection, and corneal chemical burns.

2. A method of contraception, the method comprising:
administering to a female subject a pharmaceutical composition comprising a compound according to formula I, or a pharmaceutically acceptable salt thereof, and a pharmaceutical adjuvant or a pharmaceutical carrier, 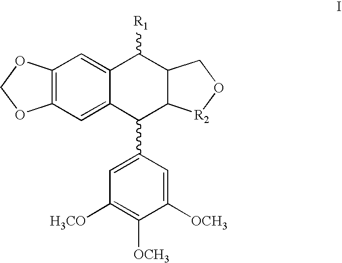
wherein the pharmaceutical composition is administered in an amount effective to reduce the rate of pregnancy in the female subject, wherein R₁ is selected from the group consisting of H, OH, and an ester group, and R₂ is selected from the group consisting of CO or CH₂.

3. The method of claim 1, wherein R₂ is CO, wherein the compound according to formula I is a compound according to formula II, 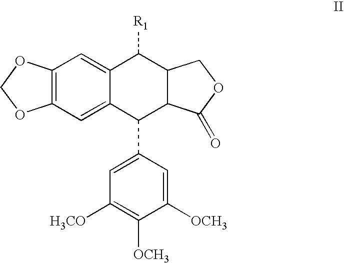
wherein the lactone ring has a cis configuration with two beta bonds, and wherein R₁ and the trimethoxyphenyl group are in alpha-position.

4. The method of claim 3, wherein the compound according to formula III is a compound selected from the group consisting of picropodophyllin and deoxypicropodophyllin.

5. The method of claim 1, wherein R₂ is CH₂, wherein the compound according to formula I is a compound according to formula III, 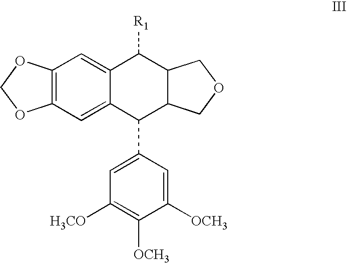
wherein the cyclo-ether ring has a cis configuration with two beta bonds, and wherein R₁ and the trimethoxyphenyl group are in alpha-position.

6. The method of claim 5, wherein the compound according to formula III is a compound selected from a group consisting of anhydropicropodophyllol and deoxyanhydropicropodophyllol.

7. The method according to claim 1, wherein R₁ is selected from the group consisting of OCOH, OCO(CH₂)₀₋₁₈CH₃, OCOCH(CH₃)₂, OCO(CH₂)₂COOH, OCOCH₂N(CH₃)₂, OCONHCH₂CH₃, OCOC₅NH₄ and OPO₃H₂.

8. The method according to claim 1, wherein the selected disease or condition is diabetes mellitus type 2 and the one or more clinical symptoms comprise elevated blood glucose levels.

9. The method of claim 1, wherein the pharmaceutical composition is administered in combination with another drug selected from the group consisting of insulin, sulfonylureas, metformin and alpha-glucosidase inhibitors.

10. The method of claim 1, wherein the selected disease or condition is nephropathy.

11. The method of claim 1, wherein the selected disease or condition is diabetic retinopathy.

12. The method of claim 1, wherein the selected disease or condition is macular degeneration and the one or more clinical symptoms comprise macular degeneration lesion area.

13. The method of claim 1, wherein the selected disease or condition is retinopathy of prematurity.

14. The method of claim 1, wherein the selected disease or condition is central retinal vein occlusion (CRVO).

15. The method of claim 1, wherein the selected disease or condition is branch retinal vein occlusion (BRVO).

16. The method of claim 1, wherein the selected disease or condition is rubeotic glaucoma.

17. The method of claim 1, wherein the selected disease or condition is thyroid eye disease.

18. The method of claim 1, wherein the selected disease or condition is corneal graft rejection.

19. The method of claim 1, wherein the selected disease or condition is corneal chemical burns.

20. The method of claim 2, wherein the compound is selected from the group consisting of picropodophyllin and deoxypicropodophyllin.

21. The method of claim 2, wherein the pharmaceutical composition is administered in combination with at least one other drug selected from the group consisting of an estrogen, a progestagen, and a prostaglandin.

22. The method of claim 2, wherein the pharmaceutical composition is administered in combination with mifepristone.

23. A method of treatment of an eye disease comprising administrating a pharmaceutical composition in combination with a physiologically acceptable carrier, wherein the pharmaceutical composition has the formula: 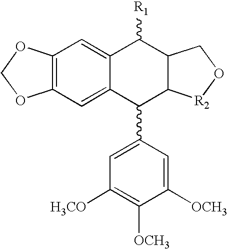
wherein R₁ is selected from the group consisting of H, OH, and an ester group, and R2 is selected from the group consisting of CO or CH₂.

24. The method according to claim 23, wherein the pharmaceutical composition is administered locally in the eye.

25. The method according to claim 23, wherein the pharmaceutical composition is administered near the eye.
